(19) United States
(12) Patent Application Publication
(10) Pub. No.: US 2023/0010962 A1
(43) Pub. Date: Jan. 12, 2023

(54) MULTIPLE STENT WITH MEMBRANE

(71) Applicant: Stental GmbH, Lörrach (DE)

(72) Inventor: Malte Neuß, Bonn (DE)

(73) Assignee: Stental GmbH, Lörrach (DE)

(21) Appl. No.: 17/785,116

(22) PCT Filed: Dec. 15, 2020
(86) PCT No.: PCT/EP2020/086262

§ 371 Date: Jun. 14, 2022

(30) Foreign Application Priority Data
Dec. 20, 2019 (DE) 10 2019 135 453.6

(51) Int. Cl. A61F 2/07

(52) U.S. Cl. CPC A61F 2/07; A61F 2002/075; A61F 2250/0063

(57) ABSTRACT

The invention relates to a multiple stent comprising at least two coaxially arranged stents (1, 3) and at least one membrane (2, 4), wherein a first stent (1) being arranged on the inside and a second stent (3) being arranged on the outside, wherein a first membrane (2) is arranged on the inside of the first stent (1) and/or a second membrane is arranged on the outside of the second stent (3), wherein the respective membrane ends (2A, 2B; 4A, 4B) are folded around the respective ends of the stent (1, 3) on which they are arranged, in such a way that the membrane ends (2A, 2B; 4A, 4B) are clamped in place between two stents (1, 3).

The invention relates to a multiple stent with at least two coaxially arranged stents and at least one membrane, wherein the ends of the membrane are fixed between two stents. The multiple stent serves in particular as a stent graft for the purpose of bridging vascular malformations, such as, for example, aneurysms or shunts, but also to reinforce unstable, fragile or thrombotic vessel walls. It is also used to reconnect vessels branching off from stented vessels.

Stent grafts for bridging vascular malformations are known in a variety of forms. As a rule, they consist of a stent that is completely or partially covered with a membrane. The membrane occludes the vascular malformation against the vessel, the stent keeps the vessel open and ensures that the membrane is in close contact with the is vessel wall.

A problem encountered with such stent grafts known from prior art involves anchoring the membrane safely to the stent. For example, although suturing the membrane to the stent framework is very reliable and durable, its provision is nonetheless also very time-consuming and for that reason expensive. Alternative developments provide for a fixation of the membrane to specific clip-like retaining elements of the stent.

Another development in this field relates to stent grafts in the form of so-called double stents, in which a membrane is arranged and held between two radially positioned stents, namely an outer and an inner stent. During the expansion of such a double stent, the membrane participates in the radial expansion and remains clamped between the two stents.

Such a double stent is known, for example, from what has been disclosed in DE 197 20 115 A1. As described hereinbefore, the known double stent provides for a single layer of material or membrane that is retained between two stents.

An alternative double stent is described in DE 10 2016 120 445 A1. In this case, a second membrane is arranged on the outer stent, wherein the membrane ends of both the first and second membrane being brought together at the ends of the stents, folded over to the inside of the inner stent and clamped under flexible tongues of the inner stent.

Basically, the known double stents are functional, but there is room for improvement in some respects.

For example, as regards the double stent first described above, the layer of material located between the two stents is subject to friction arising between the inner and outer stents during expansion, a situation which may cause damage to the material layer. In the event this damage leads to holes or cracks in the material layer immediately or only after a while of exposure, tightness problems may be the result. Therefore, an occlusion of a vascular malformation, for example, would no longer be achievable.

The variant with two membranes, which is known furthermore, suffers in particular from the disadvantage that the respective membrane ends must be clamped double-layered in the flexible tongues within the lumen of the double stent. This is not only technically demanding, but inevitably leads to a not inconsiderable accumulation of material and thus to protrusions within the stent lumen due to the projecting clamps and as well to bulging membrane ends which may form. Protrusions of this kind are predestined starting points for the formation of thrombi/clots, which can lead to stent occlusions.

With respect to the known double stents, it is, moreover, considered fundamentally disadvantageous that the inner lumen is formed in each case by a bare stent which does not have an inner cover. Turbulence is thus created in the bloodstream by the exposed struts of the stent and these must therefore be considered another target for the formation of plaque and thrombi, which may lead to an occlusion of the stent.

It is therefore the objective of the invention to present an improved stent graft that does not suffer from the disadvantages of the known double stents as pointed out hereinbefore.

It is, moreover, an object of the present invention to provide a stent graft that is easy to manufacture.

This objective is achieved by providing a multiple stent of the kind first mentioned above, which comprises at least two coaxially arranged stents and at least one membrane, wherein a first stent is arranged on the inside and a second stent is arranged on the outside and a first membrane is arranged on the inside of the first stent and/or a second membrane is arranged on the outside of the second stent. The respective ends of the membrane are wrapped around the respective ends of the stent to which they are attached in such a way that the membrane ends are clamped between two stents.

In principle, it is thus conceivable to make use of an arrangement comprising a plurality of stents and membranes arranged thereon, however the embodiments described hereinafter which comprise two or three stents and one or two membranes have essentially proven advantageous.

In the context and within the meaning of the invention, outer or outside shall be understood to denote that this part of the multiple stent is arranged closer to the vessel wall, whereas inner or inside describes a portion of the multiple stent that is arranged further away from the vessel wall and thus closer to the lumen of the multiple stent. More specific information on spatial details can also be found in the figures.

In a first preferred embodiment, the multiple stent proposed by the invention comprises an inner stent having a first end and a second end and an outer stent having a first end and a second end. The stents are arranged coaxially to each other.

In the first preferred embodiment, the inventive multiple stent further comprises an inner membrane and an outer membrane, wherein the inner membrane is disposed on the inside of the inner stent and the outer membrane is disposed on the outside of the outer stent.

The ends of the membranes are folded around the ends of the stents on which they are arranged in such a way that the respective ends of the membranes are clamped in place between the stents.

In a second preferred embodiment, the multiple stent proposed by the invention comprises an inner stent having a first end and a second end and an outer stent having a first end and a second end. The stents are arranged coaxially to each other.

In the second preferred embodiment, the multiple stent according to the invention further comprises an inner membrane, with the inner membrane being arranged on the inside of the inner stent.

The ends of the inner membrane are folded around the ends of the inner stent such that the ends of the inner membrane are clamped in place between the stents.

In a third preferred embodiment, the multiple stent proposed by the invention comprises an inner stent having a first end and a second end and an outer stent having a first end and a second end. The stents are arranged coaxially to each other.

In the third preferred embodiment, the multiple stent according to the invention further comprises an outer membrane, with the outer membrane being arranged on the outside of the outer stent.

The ends of the outer membrane are folded around the ends of the outer stent such that the ends of the outer membrane are clamped in place between the stents.

In a variant of the embodiments referred to as preferred, the multiple stent in each case consists of an additional third or middle stent which is disposed between the inner stent and the outer stent.

The middle stent can be provided to stabilize the multiple stent; however the middle stent may also or additionally be provided with a view to further securing the membranes. To enhance the fixation of the membranes, the middle stent may, for example, have an appropriate surface structure or be made of materials that counteract slippage of the membranes. For this purpose, it is also conceivable for such surfaces and materials of the middle stent to consist, for example, of slightly roughened surfaces to increase frictional forces, which would not be advantageous for the inner and outer stents, because these are, at least in certain embodiments, in direct contact with the vessel wall or the flow of blood.

In accordance with an additional variant of the embodiments described, it is conceivable for certain stents of the multiple stent, i.e. for example the inner and/or the middle and/or the outer stent, not to be provided in the form of a uniform or continuous stent body, but to be composed of a plurality of, as the case may be, connected or unconnected stent elements (i.e. individual stent bodies). In their entirety, these stent elements may correspond to the length of the multiple stent or to the length of the other stents of the multiple stent, however, they may also be shorter in total, so that they create gaps relative to the other stents, or longer so that they create overlaps.

For purposes of this additional variation, terms such as “inner stent” or “outer stent” are more appropriately understood as “inner stent position” or as “outer stent position” in the sense that they refer to a particular location within the multiple stent, which may be occupied by a single or continuous stent body or, alternatively, by a plurality of connected or disconnected stent elements.

Preferably, at least one stent of the multiple stent is provided in this case from a continuous or coherent stent body. This is preferably the stent on which the membrane is arranged.

The following elaborations apply to all embodiments of the multiple stent proposed by the invention and, in particular, to the preferred embodiments and to the variants that have been outlined.

Any biological or artificial material suitable for the purpose can be employed for the membranes. Usually, the membranes consist of plastic material, preferably a plastic tube, which is pulled over the respective stent. For example, a suitable material is polytetrafluoroethylene, PTFE, especially ePTFE, which has the elasticity required for the expansion process. Other plastics unobjectionable from a medical viewpoint, such as polyester, polyolefins, polyurethanes, polyurethane carbonate and the like, may also be employed. Also conceivable, for example, are membranes knitted, woven or sewn from threads, especially polymer threads.

The length of the membranes may be selected such that their ends overlap between the stents after folding over. Preferably, the membranes are then 5 to 25%, more preferably 25 to 50%, further more preferably 50 to 75%, and especially 75 to 100% longer than the stent on which they are placed.

However, embodiments are also conceivable in which the ends of at least one membrane overlap, meaning, the membrane is thus more than twice as long as the stent on which it is arranged.

The membranes, insofar as more than one is arranged in the multiple stent, may be made of different materials and have different lengths.

When several membranes are provided, they can be of the same length or have different lengths. The membranes may be arranged symmetrically or be provided in some other configuration.

The stents may be balloon-expandable or self-expandable. Furthermore, the stents can be braided or cut from a tube of suitable diameter using a laser cutting technique. They are provided with a mesh or lattice structure.

The multiple stent principle in accordance with the invention inherently leads to a relatively high wall thickness of the construct, which may impose restrictions on the maneuverability in a patient's vascular system. This can be countered by choosing a low wall thickness of the tubes from which the stents are cut or a small diameter of the wires used for braiding of the stents, for example in the range of between 0.05 and 0.50 mm, preferably between 0.10 and 0.20 mm and in particular approx. 0.15 mm. The web width as well can be reduced, for example, to between 0.05 and 0.50 mm, preferably between 0.10 and 0.20 mm and in particular to approx. 0.15 mm. By making use of at least two stents, a high radial force is achieved even with low wall thicknesses or wire diameters.

Stents may generally be fabricated of customarily known materials, for example of medical steel, cobalt-chromium alloys and nickel-titanium alloys or optional combinations thereof. Plastics (polymers), for example resorbable plastic materials, including various polylactates as they are known from the state of the art, may also is be employed, as well as combinations consisting of metal stents and plastic stents.

In this respect, the arrangement and the materials to be used for the stents are chosen by those skilled in the art to suit the respective applications at hand. For example, the inner stent may thus comprise a metal and the outer stent may comprise a plastic, or vice versa. However, the all the stents may as well be made of the same material.

It shall be understood that the different stents may have different thicknesses and be of different lengths, with at least the inner stent and the outer stent of the inventive multiple stent preferably being arranged symmetrically with respect to each other.

However, also conceivable are embodiments according to which the stents are arranged offset from one another or are of different lengths, insofar as this ensures the required clamping effect to be still achieved.

Likewise, different designs may be employed for the stents. It is thus preferred, for example, to provide the stents that are located further to the outside or the outer stent with smaller meshes than stents located further to the inside, or the inner stent. In this way, a compressive stress is created during expansion, which has an advantageous effect on the radial force and the coherence of the construct. This ensures that high strength and durability of the construct are achieved.

The nominal diameter of the more inwardly located stents or the inner stent is at least as large as that of the more outwardly arranged stents, although the nominal diameter of the more inwardly located stents should preferably be chosen to be even slightly larger than that of the more outwardly arranged stents in order to achieve a greater clamping effect.

Clamping the ends of the membrane in place between the stents results in a reliable anchoring of the membrane and can be easily produced, since there is no need to fabricate and operate small-scale clamping elements or perform sophisticated and elaborate suturing work.

In a further development of the multiple stent proposed by the invention, said stent is particularly suitable for use in multilumen implants. A multilumen implant is defined as such an implant that is intended for implantation in the vascular system and comprises branches to be adapted to the vascular morphology of the patient. As a rule, these are multilumen stent grafts.

Multilumen implants are often offered in the form of kits because individual vascular morphology can vary significantly from patient to patient and customized designs and fabrication are expensive and time-consuming. In this context, during or in advance of the intervention, the individual branches of the multilumen implant are chosen to match the respective diameters of the vessels to be treated. A critical factor in this respect is that the individual components must be connected securely and easily.

Accordingly, the further development provides for the retaining elements to be arranged at least at one end or at an edge region of the multiple stent, with the multiple stent being secured in a multilumen implant by said elements. For example, the retaining elements can be provided in the form of hook-shaped and radially outwardly oriented elements. Said hook-shaped and radially outwardly oriented elements can preferably be part of the bare stent, that is, a stent which is not covered with a membrane.

Certain remolding or reshaping of the stent ends or the edge regions of the stents may also serve as retaining elements. It is conceivable, for instance, that at least one end portion of at least one stent that forms part of the multiple stent expands outwardly. For this purpose, the terminally arranged struts of the stent can be specially shaped, for example, and in particular they can be made longer than the other struts to promote such a reshaping.

It is also thinkable that in particular the hook-shaped retaining elements are provided so as to perform a dual function in that they are provided on the covered stent and, aside from their retaining function in the multilumen implant, enable the membrane to be additionally secured at the folding-over point. In any case, the retaining elements must be arranged such that they protrude sufficiently far from the multiple stent to allow adequate anchorage in a multilumen implant.

To achieve this, the end of the stent provided with retaining elements can, for example, be slightly longer and thus already protrude from the multiple stent, which is in particular expedient if one of the inner stents is equipped with retaining elements.

In the event that the outer stent is equipped with hook-shaped retaining elements, it is preferred, especially with regard to the second embodiment described, to arrange for the uncovered outer stent to be shorter in overall length than the inner covered stent, resulting in the outer stent to be completely covered with respect to the vessel lumen by the inner stent or by the membrane provided on this stent.

Aside from the hooks mentioned here, other designs of the retaining elements are conceivable, for example also those that are specially adapted to relevant connecting points in the multilumen implant, similar to a key-lock principle or a hook-eye principle.

The combination of an inner and an outer stent with an outer and/or an inner membrane results in a surprisingly stable implant to be achieved that nevertheless offers a high degree of flexibility. The membranes and the at least double stent configuration contribute to stabilizing the multiple stent and allow the stent walls to be maintained comparatively thin. Nevertheless, the multiple stent exhibits good radial force.

Further elucidation of the invention is provided through the enclosed figures showing preferred embodiments of the invention. It goes without saying that the characteristics shown in the figures shall in each case be regarded individually as being part of the invention and should not be understood exclusively in the context of the other characteristics illustrated in the figures, where

FIG. 1 shows the schematic structure of a first embodiment of the multiple stent according to the invention;

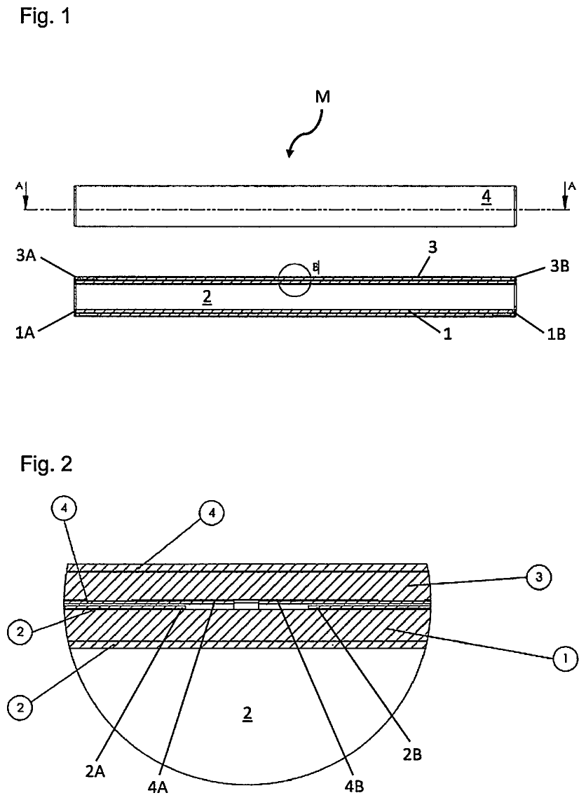

FIG. 2 shows detail B of FIG. 1 illustrating the arrangement of the stents and membranes according to the first embodiment of the inventive multiple stent;

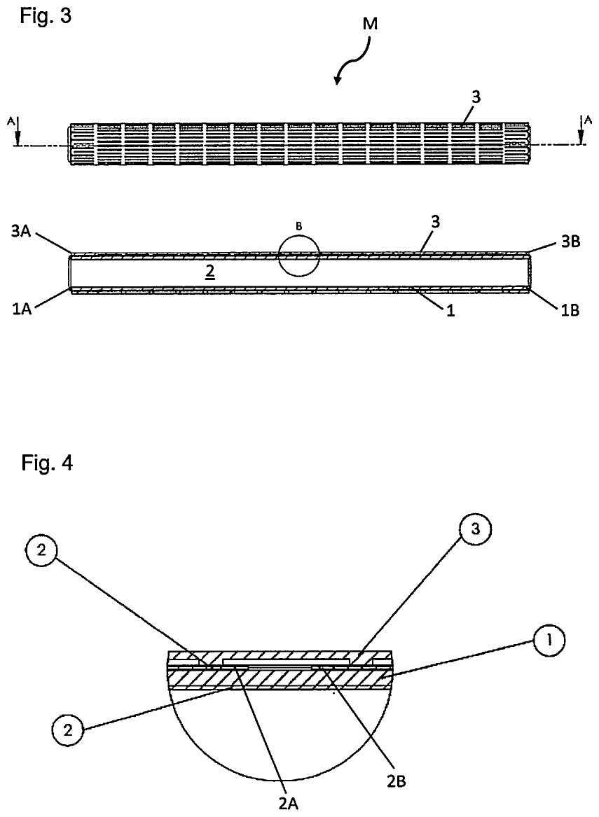

FIG. 3 shows the schematic structure of a second embodiment of the multiple stent according to the invention;

FIG. 4 shows detail B of FIG. 3 illustrating the arrangement of the stents and the membrane according to the second embodiment of the inventive multiple stent;

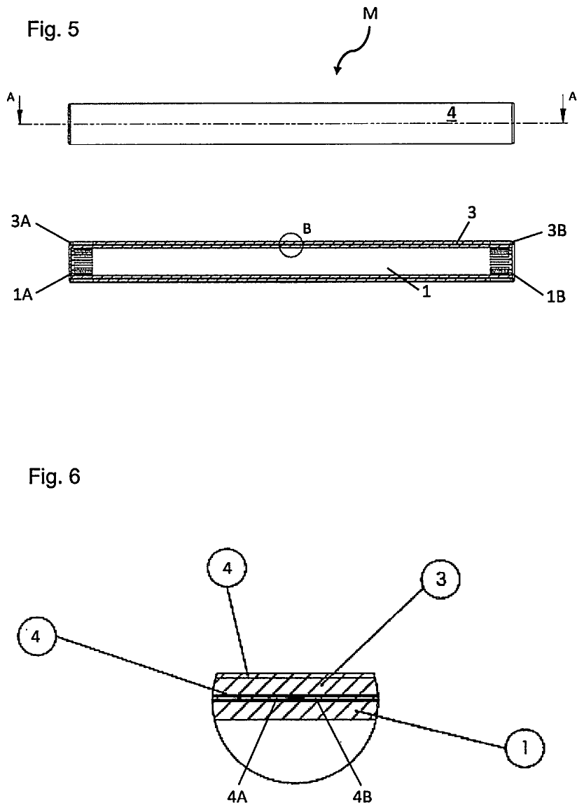

FIG. 5 shows the schematic structure of a third embodiment of the multiple stent according to the invention;

FIG. 6 shows detail B of FIG. 5 illustrating the arrangement of the stents and the membrane according to the third embodiment of the inventive multiple stent;

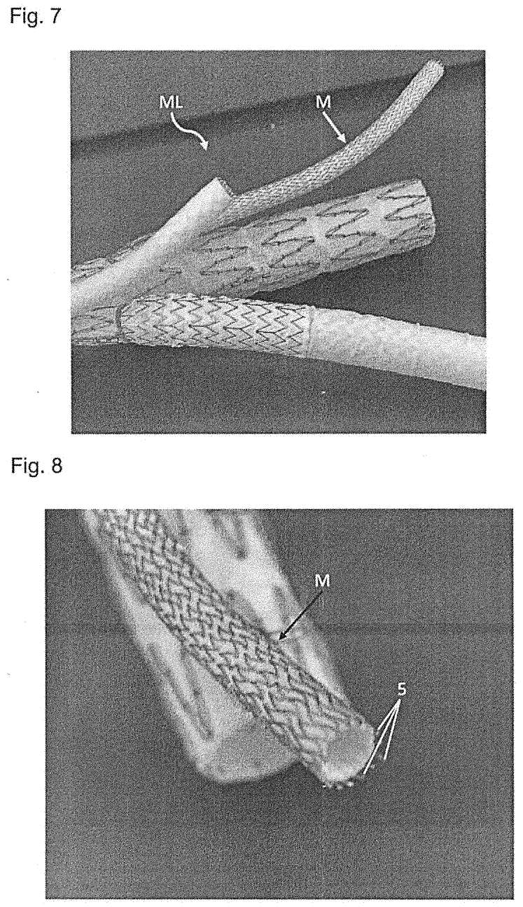

FIG. 7 illustrates the application of the multiple stent according to the invention in a multilumen implant;

FIG. 8 is a detailed view of a further development of the inventive multiple stent provided with retaining elements for use in a multilumen implant;

FIG. 1 shows a first embodiment of the multiple stent M according to the invention with an inner stent 1 having a first end 1A and a second end 1B and an outer stent 3 having a first end 3A and a second end 3B, wherein the stents 1, 3 are arranged coaxially to each other. The multiple stent M is shown in the non-expanded state. An is inner membrane 2 is arranged on the inside of the inner stent 1 and an outer membrane 4 is arranged on the outside of the outer stent 3.

Membranes 2, 4 are folded around the ends 1A, 1B, 3A, 3B of the stents 1, 3 causing them to be clamped with their ends between the stents 1, 3, respectively.

The stents 1, 3 may be provided so as to be balloon-expandable or self-expandable, with all known materials being suitable for use. The stents 1, 3 can be fabricated from different materials. The nominal diameter of the inner stent 1 is at least as large as that of the outer stent 3; preferably, the nominal diameter of the inner stent 1 should even be chosen to be slightly larger than that of the outer stent 3 to bring about a higher clamping effect.

The membranes 2, 4 can be made of the widest variety of known materials, but with tubular membranes made of ePTFE being preferred. The membranes 2, 4 can also be provided from different materials.

FIG. 2 shows a detailed view B of FIG. 1 as a closer view of the arrangement of the stents 1, 3 and the membranes 2, 4 relative to each other. The inner membrane 2 and the outer membrane 4 are folded over with their two ends 2A, 2B, 4A, 4B respectively around the end 1A, 1B, 3A, 3B of the stent 1, 3 on which they rest inside and outside, respectively, in the area between the stents 1, 3. The ends 2A, 2B, 4A, 4B or more precisely the end regions of the membranes 2, 4 are thus clamped in place between the two stents 1, 3. As shown in the figure, the membranes 2, 4 can be of different lengths, but they can also be provided with the same length. The length of the membranes 2, 4 may be selected, as illustrated, such that the respective ends 2A, 2B or 4A, 4B between the stents 1, 3 do not touch or overlap, however the length of the membranes 2, 4 may also be selected such that the respective ends 2A, 2B or 4A, 4B between the stents 1, 3 touch or overlap.

FIG. 3 shows a second embodiment of the multiple stent M according to the invention with a first inner stent 1 having a first end 1A and a second end 1B and a second outer stent 3 having a first end 3A and a second end 3B, wherein the stents 1, 3 are arranged coaxially to each other. The multiple stent M is shown in the non-expanded state. In this embodiment, only one membrane 2 is arranged on the inside of the inner stent 1.

The membrane 2 is folded around the ends 1A, 1B, of the inner stent 1 causing the membrane to be clamped with their ends between the two stents 1, 3.

The stents 1, 3 may be provided so as to be balloon-expandable or self-expandable, with all known materials being suitable for use. The stents 1, 3 can be fabricated from different materials. The nominal diameter of the inner stent 1 is at least as large as that of the outer stent 3; preferably, the nominal diameter of the inner stent 1 should even be chosen to be slightly larger than that of the outer stent 3 to bring about a higher clamping effect.

The membrane 2 can be made of the widest variety of known materials, but with tubular membranes made of ePTFE being preferred.

FIG. 4 shows a detailed view B of FIG. 3 as a closer view of the arrangement of the stents 1, 3 and the membrane 2 relative to each other. The inner membrane 2 is folded over with its two ends 2A, 2B respectively around the end 1A, 1B of the inner stent 1 into the area between the stents 1, 3. The ends 2A, 2B of the membrane 2 are thus clamped in place between the two stents 1, 3. The length of the membrane 2 may be selected, as shown, such that its ends 2A, 2B between the stents 1, 3 do not touch or overlap; however, the length of the membrane 2 may also be selected such that its ends 2A, 2B between the stents 1, 3 touch or overlap.

FIG. 5 shows a third embodiment of the multiple stent M according to the invention with a first inner stent 1 having a first end 1A and a second end 1B and a second outer stent 3 having a first end 3A and a second end 3B. The stents 1, 3 are arranged coaxially to each other. The multiple stent M is shown in the non-expanded state. In this embodiment, only one membrane 4 is arranged on the outside of the outer stent 3.

The membrane 4 is folded around ends 3A, 3B, of the outer stent 3 causing the membrane to be clamped with their ends between the two stents 1, 3.

The stents 1, 3 may be provided so as to be balloon-expandable or self-expandable, with all known materials being suitable for use. The stents 1, 3 can be fabricated from different materials.

The nominal diameter of the inner stent 1 is at least as large as that of the outer stent 3; preferably, the nominal diameter of the inner stent 1 should even be chosen to be slightly larger than that of the outer stent 3 to bring about a higher clamping effect.

The membrane 4 can be made of the widest variety of known materials, but with tubular membranes made of ePTFE being preferred.

FIG. 6 shows a detailed view B of FIG. 5 as a closer view of the arrangement of the stents 1, 3 and the membrane 4 relative to each other. The outer membrane 4 is folded over with its two ends 4A, 4B respectively around the end 3A, 3B of the outer stent 3 into the area between the stents 1, 3. The ends 4A, 4B of the membrane 4 are thus clamped in place between the two stents 1, 3.

The length of the membrane 4 may be selected, as shown, such that its ends 4A, 4B between the stents 1, 3 do not touch or overlap; however, the length of the membrane 4 may also be selected such that its ends 4A, 4B between the stents 1, 3 touch or overlap.

FIG. 7 shows the application of a further development of multiple stent M proposed by the invention for use in a multilumen implant ML, i.e. in a branched stent graft.

FIG. 8 shows in detail the end region of a further development of the multiple stent M according to the invention for use in a multilumen implant ML as illustrated in FIG. 7. Said further development provides for the retaining elements 5 to be arranged at least at one end of the multiple stent M; by means of said elements multiple stent M can be secured in a multilumen implant ML. Multilumen implants ML are often offered in the form of kits, with the individual branches of the multilumen implant ML being selected to match the respective diameters of the vessels to be treated. Critical in this context is the connection of the individual components. With this in mind, the further development preferably provides for the retaining elements 5 to be composed of hook-shaped and radially outwardly directed elements 5. Preferably, these can be part of the uncovered stent, because the ends of the covered stent are covered by the folded-over membrane. A piercing of the membrane by the retaining elements would result in unnecessary material damage. It is also conceivable that the retaining elements 5 are provided so as to perform a dual function in that they are provided on the covered stent and, aside from their retaining function in the multilumen implant ML, enable the membrane to be additionally secured at the folding-over point. In any case, the retaining elements 5 must be arranged such that they protrude sufficiently far from the multiple stent to allow adequate anchorage in a multilumen implant.

### LIST OF REFERENCE NUMERALS

1 inner (first) stent (1A, 1B: ends of the inner stent)
2 inner (first) membrane (2A, 2B: ends of the inner membrane)
3 outer (second) stent (3A, 3B: ends of the outer stent)
4 outer (second) membrane (4A, 4B: ends of the outer membrane) retaining elements
M multiple stent
ML multilumen implant

1. Multiple stent comprising at least two coaxially arranged stents (1, 3) and at least one membrane (2, 4), wherein a first stent (1) being arranged on the inside and a second stent (3) being arranged on the outside, characterized in that a first membrane (2) is arranged on the inside of the first stent (1) and/or a second membrane (4) is arranged on the outside of the second stent (3), wherein the respective membrane ends (2A, 2B; 4A, 4B) are folded around the respective ends (1A, 1B; 3A, 3B) of the stent (1, 3) on which they are arranged, in such a way that the membrane ends (2A, 2B; 4A, 4B) are guided between the first and the second stent (1, 3).

2. Multiple stent according to claim 1, characterized in that the more inwardly arranged stents (1) have a nominal diameter of at least the same size as the more outwardly arranged stents (3), wherein the more inwardly arranged stents (1) preferably have a larger nominal diameter than the more outwardly arranged stents (3).

3. Multiple stent according to claim 1, characterized in that the at least one membrane (2, 4) is provided in tubular form.

4. Multiple stent according to claim 1, characterized in that the at least one membrane (2, 4) is 5 to 25% longer, preferably 25 to 50% longer, further preferably 50 to 75% and in particular 75% to 100% longer than the stent (1, 3) on which it is arranged.

5. Multiple stent according to any one of the preceding claims claim 1, characterized in that the at least one membrane (2, 4) is more than twice as long as the stent (1, 3) on which it is arranged, so that the folded-over membrane ends (2A, 2B; 4A, 4B) overlap between the stents (1, 3), respectively.

6. Multiple stent according to claim 1, wherein the membranes (2, 4) may be made of a film or provided knitted, woven or sewn from a thread.

7. Multiple stent according to claim 1, comprising two coaxially arranged stents (1, 3) and two membranes (2, 4), with the first stent (1) being provided on the inside and the second stent (3) being provided on the outside, characterized in that the first membrane (2) is arranged on the inside of the first stent (1) and the membrane ends (2A, 2B) of the first membrane (2) are folded over to the outside of the first stent (1) and the second membrane (4) is arranged on the outside of the second stent (3) and the membrane ends (4A, 4B) of the second membrane (4) are folded over to the inside of the second stent (3) so that the membrane ends (2A, 2B; 4A, 4B) are clamped in place between the stents (1, 3).

8. Multiple stent according to claim 1, comprising two coaxially arranged stents (1, 3) and one membrane (2), wherein the first stent (1) is provided on the inside and the second stent (3) is provided on the outside, characterized in that the membrane (2) is arranged on the inside of the first stent (1) and the membrane ends (2A, 2B) are folded over onto the outside of the first stent (1) so that the membrane ends (2A, 2B) are clamped in place between the stents (1, 3).

9. Multiple stent according to claim 1 with two coaxially arranged stents (1, 3) and one membrane (4), wherein the first stent (1) is provided on the inside and the second stent (3) is provided on the outside, characterized in that the membrane (4) is arranged on the outside of the second stent (3) and the membrane ends (4A, 4B) are folded over onto the inside of the second stent (3) so that the membrane ends (4A, 4B) are clamped in place between the stents (1, 3).

10. Multiple stent according to claim 1, comprising three coaxially arranged stents and two membranes, wherein the first stent being arranged on the inside, the second stent being provided on the outside, and the third stent being provided between the first stent and the second stent, characterized in that the first membrane is arranged on the inside of the first stent and the membrane ends of the first membrane are folded over to the outside of the first stent and are clamped in place between the first and the third stent and the second membrane is arranged on the outside of the second stent and the membrane ends of the second membrane are folded over to the inside of the second stent and are clamped in place between the second and the third stent.

11. Multiple stent according to claim 1, wherein retaining elements (5) are provided at least at one end of a stent of the multiple stent (M).

12. Multiple stent according to claim 1, wherein the retaining elements (5) are provided as an extension of at least one end portion of at least one stent of the multiple stent (M).

13. Multiple stent according to claim 1, wherein the retaining elements (5) are provided to be of hook-shaped configuration and directed radially outward.

14. Multiple stent according to claim 1, characterized in that at least one stent of the multiple stent (M) comprises a plurality of individual stent elements.
